(19) United States
(12) Patent Application Publication
(10) Pub. No.: US 2025/0017754 A1
(43) Pub. Date: Jan. 16, 2025

(54) Smart Custom Orthotic

(71) Applicant: Fiomet Ventures, Inc., Cincinnati, OH (US)

(72) Inventors: Scott J. Rapp, Cincinnati, OH (US); Christopher B. Gordon, Cincinnati, OH (US); Horacio L. Rilo, Cincinnati, OH (US); Jared Sartee, Pemberton (CA)

(21) Appl. No.: 18/894,724

(22) Filed: Sep. 24, 2024

Related U.S. Application Data
(63) Continuation-in-part of application No. 17/159,242, filed on Jan. 27, 2021, now abandoned.
Application No. 17/159,242 is a continuation-in-part of application No. 15/289,071, filed on Oct. 7, 2016, now Pat. No. 10,973,413.
(60) Provisional application No. 62/969,067, filed on Feb. 1, 2020.
(60) Provisional application No. 62/238,522, filed on Oct. 7, 2015.

(51) Int. Cl. A61F 5/058

(52) U.S. Cl. CPC A61F 5/05891

(57) ABSTRACT

A portable customizable smart orthotic for wound care, trauma and critical care. The cranial orthotic includes an exterior shell, with or without an accompanying bladder providing additional cushion to the wound environment. The cranial orthotic is adapted to sense pressures and biometrics associated with the wound environment and as well as heat or cool portions of the wound environment. A communications module accompanies the smart cranial orthotic.

This application claims priority to application Ser. No. 17/159,242—Smart Custom Orthotic—, filed Jan. 27, 2022 that claimed the benefit of Provisional Application 62/969,067—Wound Treatment Devices—filed Feb. 1, 2020 and priority to U.S. application Ser. No. 15/289,071—Advanced Compression Garments and Systems—, filed Oct. 7, 2016 (now U.S. Pat. No. 10,973,413 Patent-Granted Apr. 13, 2021) that claimed the benefit of U.S. Provisional Application 62/238,522, filed on Oct. 7, 2015.

### BACKGROUND OF THE INVENTION

### A. Field of the Invention

The current smart custom orthotic is a portable smart helmet, cap or splint device utilized for custom orthotics, wound care, trauma and critical care. Features of the current invention can reduce post injury trauma and adverse physiological events while also providing notifications that can assist medical intervention. Among other things, the smart custom cranial orthotic can be adapted to customize pressure applied to the wound or abnormal environment.

It is estimated that over 13 million living people have symptoms related to traumatic brain injury in the US and Europe populations alone¹. Worldwide, the incidence is increasing due to the more readily available methods of motor transportation such as motorcycles and low-cost scooters. Even when helmeted, the transmitted force to the brain can lead to concussions, traumatic brain injury, to diffuse axonal injury (DAI) and death. It has been reported that over 20% of all concussions are sports-related in the US².

As such, multiple efforts are focused on improving the materials in both sports-related and commercial helmets for protection. The ability to disperse forces through the helmet structures may reduce the harmful impact of accelerations and decelerations on the brain movement within the calvarium. The geometric structure and substance of the padding portion of the helmet (i.e. prismatic lattice) have been shown to reduce TBI³.

Specific focus has been on custom liners for helmets such as viscoelastic polymers which have been shown to reduce strain and strain-rates for head impact¹. Penetrating brain injuries carry the highest mortality rates⁴. Retrospective analysis of the Iraq and Afghanistan wars revealed that numbers of penetrating TBIs exceeded closed TBIs by a ratio of 2:1 and 1.3:1 respectively⁵. The ability to control hemostasis with penetrating brain injuries in the field is quite difficult. Too much pressure can cause tonsillar herniation or brain injury and death, too little compression may result in excessive bleeding and shock. It is clear there is a need for specific wound care solutions in these scenarios.

In penetrating head trauma, closed head injuries and acquired cranial deformities from surgical intervention, multiple adverse clinical sequalae may result. The scalp provides a robust blood supply to the surrounding anatomic structures and disruption resulting from insult can lead to excessive blood loss and even death within a short period of time. Obtaining hemostasis is quite feasible in the operating theater or ambulatory medical setting, however in the field and away from medical help, scalp injuries may be difficult to control leading to hypotension, shock and death. Additionally, penetrating injuries to the scalp and underlying skull represent an even more severe clinical scenario where serious sequalae such as hemorrhagic shock, hypotension, hypothermia and low perfusion pressures can lead to further tissue damage and death. Lack of cerebral perfusion leads to hypoxemia and neuronal degradation. Hypotension and hypothermia in the face of a traumatic event have been shown to be critical contributing factors that lead to higher rates of secondary wound infections and significant downstream comorbidities.

Swelling from the above injuries can be rather profound at the time of injury, but may peak at 48 hours from injury. Compressive bandages are a first line treatment defense against hemorrhage. However, static application of the bandages without manipulation over the course of a physiologic response to injury may result in further injuries. Dressings applied at the initial time of injury may become too tight and in itself cause further injury or harm to the individual if variations to inflammation and swelling occur. Alternatively, hypobaric environments, such as during transport in flight also results in changes to both the perfusion pressures to the tissues as well as risk of wound infection.

Selective brain cooling has been shown to provide neuroprotective capabilities after brain trauma. Induced hypothermia has been shown to minimize acute brain damage in animal models after penetrating ballistic-like brain injury (PBBI). These models have shown improved preservation of axonal integrity, cellular apoptosis, blood-brain barrier disruption, neuroinflammation and improved behavior outcomes both in TBI and PBBI. Reducing brain temperature by 2-3 degrees Celsius while maintaining corporal normothermia may have life-saving capabilities. Selectively cooling the brain may also allow for a greater time window to allow for transportation from the field to the medical facility where more effective strategies to treat repairable injuries may be obtained. However, focal cooling of the brain both in the field and in the medical arena without causing systemic lowering of temperature has been difficult to achieve in clinical practice. As above, hypothermia has been shown to be a critical factor in overall mortality, wound infection rates, blood coagulopathies, and overall hospital stay following severe traumatic injury.

### B. Description of the Previous Art

Any discussion of references cited herein merely summarizes the disclosures of the cited references. Applicant makes no admission that any cited reference or portion thereof is relevant prior art. Applicant reserves the right to challenge the accuracy, relevancy and veracity of the cited references. Publications or presentations that may indicate a state-of-art include:
1. Siegkas P, Sharp D J, Ghajari M. The traumatic brain injury mitigation effects of a new viscoelastic add-on liner. Sci Rep. 2019 Mar. 5; 9 (1): 3471.
2. National Center for Injury Prevention and Control. Report to Congress on mild traumatic brain injury in the United States: steps to prevent a serious public helath problem. Atlanta, GA: Centers for Disease Control and Prevention, National Center for Injury Prevention and Control, 2003
3. Khosroshahi S F, Duckworth H, Galvanetto U, Ghajari M. The effects of topology and relative density of lattice liners on traumatic brain injury mitigation. J Biomech. 2019 Dec. 3; 97:109376.
4. Fathalla H, Ashry A, El-Fiki A. Managing military penetrating brain injuries in the war zone: lessons learned. Neurosurg Focus. 2018 Dec. 1; 45 (6): E6.
5. Orman J A, Geyer D, Jones J, Schneider E B, Grafman J, Pugh M J, Dubose J. Epidemiology of moderate-to-severe penetrating versus closed traumatic brain injury in the Iraq and Afghanistan wars. J Trauma Acute Care Surg. 2012 December; 73 (6 Suppl 5): S496-502.

### SUMMARY OF THE INVENTION

The present custom cranial orthotic device and system can address adverse events following trauma and can preserve brain hemodynamics, brain metabolism and neurobehavior during subacute injury period

The current device and system embodiments are directed towards a custom cranial orthotic that can provide real time biometric feedback from multiple different sensors. Therapeutic interventions such as hemostasis, compression, adaptive heating and cooling and improved wound care optimization are possible with the custom cranial orthotic device and system.

To address hemorrhage following injury, the orthotic has a custom foaming capability to adapt to any contour irregularity such as soft tissue scalp injury to even a concavity or convexity due to bony skull malposition. The foaming agents are able to expand into the defect or around the defect to provide focal compression and improved hemostasis. By providing diffuse pressure over a greater surface area in and around the wound, less pressure can be used to obtain hemostatic control. With penetrating injuries and concavities to the skull, the foam will expand into this area providing a more appropriate mold of the defect rather than conventional dressings that are difficult to contour in the face of a dirty or bleeding wound. A bladder interposition made of polyurethane or other polymeric materials may be implemented to ensure a watertight pressure application and enhanced rigidity to the foamed dressing.

Outside the foaming agents, a hard-collapsible shell can be applied. The shell is collapsible for ease of storage and transport on the individual or field equipment (i.e. ambulance or military vehicle). The composition of the shell will be strong enough that it can reduce against further traumatic events. The shell is also adjustable to provide finer and evenly distributed compression. Leaflets associated with the helmet provide control of external compression (more or less) supplied by the helmet or cap as required by medical parameters or when indicated by pressure sensors.

Within the scope of the current invention, biometric sensors can be positioned in the bladder, the outer shell or both. In select embodiments of the helmet or cap, biometric sensors such as pressure sensors can be place at multiple locations of the invention. Use of one or more pressure sensors can provide alerts indications and/or alerts of acceptable, excessive or insufficient pressures. Insufficient pressures may result in excessive hemorrhage, low cerebral perfusion pressures, secondary hypoxia and neurodegeneration such as axonal or glial death. Excessive pressures may result in brain herniation, soft tissue ischemia, cerebral vascular injury or stroke, and death. A biometric sensor communicates with communications module adapted to communicate with a computer distinct from the communications module. Communications module includes a transmitter or a transceiver allowing wireless communications with a remote computer that provides real real-time calculation/determination of the biometric(s) sensed. Communication modules can also include a memory, a microprocessor or processor and software (computer component) for calculating the biometric measurements sensed by the biometric sensors and controlling the helmet's heat/cooling semiconductors. Depending on medical treatment parameters, the remote computer or the communications module's computer module can be used to calculate sensed biometrics and control the heat/cooling semiconductors. In accordance with the present invention, the computer or computer module can calculate biometrics, including but not limited to, pressure, temperature, blood pressure, lactate levels, pH, sodium levels, potassium level, glucose levels, apoptotic factors, nitric oxide (NO) and SVO2 levels sensed by their respective sensors.

In selected embodiments of the current invention, semiconductors, such as micro-Peltier coolers, can be included in the bladder, the outer shell, foam or any combination to provide heating or cooling. The direction of the current flow through micro-semiconductors allows for either cooling or warming. Power for the semiconductors and other components of the current orthotic can be provided by batteries, a connection with an alternating current power source or a radio frequency energy supply. Use of carbon nanotubes can improve performance of the semiconductors.

It is believed that use of micro-semiconductors or semiconductors allows the application of different voltages and current across two distinct semiconductors, thereby generating a “heat flux” of hot or cold across the semiconductors' interfaces. Direction of current crossing the interfaces changes the generated heat flux from hot to cold or cold to hot such that the change the direction of current can result in a 25 degree Celsius deferential of temperature proximate the interfaces. In select preferred embodiments, micropeltier devices can be utilized to generate the temperature deferential.

The helmet shell and bladder can be segmented to allow collapsibility for ease of storage. Within the scope of the current invention, the outer shell can be oriented to fit around other body parts, limbs, torso or hands. Cured foams associated with the present invention can be open cell that allow heated or cooled air travel throughout the apparatus. Tunnels in the foams can also be created during curing of the created foam. Hydrogels or equivalent resorbables can create channels in the cured foam by evaporating spontaneously or over time creating channels to facilitate movement of heated or cooled air. It is believed that assisting the circulation of cold or hot air at preselected temperatures can improve healing of the tissue and viability of tissue. By way of illustration, induction of localized hypothermia can be utilized to protect tissue. Cooling the brain after traumatic injury can reduce metabolic activity which can reduce further apoptosis, harmful metabolite formation and further tissue injury.

The current custom orthotic apparatus also has application in veterinary medicine. Custom foaming devices such as helmets and splints can be configured with external hard shells and adapted to treat and protect animal injuries from trauma or during the post-operative period for animals. Those animals include, but are not limited to equine, canine, feline or zoo animals.

An aspect of the present invention is to provide a customized orthotic.

Another aspect of the present invention is to provide an orthotic that is controlled by the orthotic's communications module or via wireless transmission from a computing device remote from the communications module.

Yet another aspect of the present invention is to provide an orthotic that provides adjustable pressures to different zones of the orthotic.

Still another aspect of the present invention is to provide sensors for the orthotic adapted to sense pressure and other biometrics.

Yet still another aspect of the present invention is to provide a customized orthotic including a bladder contacting the wound environment.

Another aspect of the present invention is to provide a customized cranial orthotic.

Still another aspect of the present invention is to provide collapsible shell allowing for easier transport of orthotic.

Yet still another aspect of the present invention is to provide an orthotic for use about the skull.

Still another aspect of the present invention is to provide an orthotic for use about a limb or other body part.

Another aspect of the present invention is to provide an orthotic for human or veterinary usage.

Still another aspect of the present invention is to utilize semiconductors to assist in control the temperatures of the wound environment.

Yet still another aspect of the present invention is to utilize cured foam that enhances air movement about the wound environment.

A preferred embodiment of the current invention can be described as a cranial orthotic (20) comprising: a) a bladder (40) positioned proximate to a wound environment (10) and fitted to contact a contour of a portion of an inward side (23) of an unfolded collapsible exterior shell (22) of the cranial orthotic (20); the bladder (40) comprising: i) a first expansion fluid (36f) mixed with a second expansion fluid (36s) generating a foam volume and a gas supplying a preselected pressure to the wound environment (10); ii) micropores (42) regulating the volume of the gas inside the bladder; and iii) a plurality of polymerized beads (330); b) the unfolded collapsible exterior shell (22) comprising: i) a plurality of interconnected rotatable sections (24); each rotatable section (24) comprising a first aperture (25f) connected to a first tie (26f) and a second aperture (25s) connected to the second tie (26f) such that the plurality of interconnected rotatable sections (24) is collapsible about itself when the cranial orthotic (20) is folded; ii) a removable vent (28) providing ventilation for the wound environment (10) and an access to a communications junction (56); and iii) one or more ports (200) extending through the exterior shell (22); the one or more ports (200) adapted to transport the expansion fluids (36f, 36s) to the bladder (40); c) a three dimensional array (48) conforming with the contour of the portion of the inward side (23) of the unfolded collapsible exterior shell (22) and connected to an inward side (23) of the bladder (40); the three dimensional array comprising: i) a group of first sensors (50f), wherein the group of first sensors (50) are adapted to sense pressures associated with the wound environment (10); and ii) the communications junction (56) comprising a first magnetic field (58); d) a plurality of semiconductors (60) connected to an inward side (45) of the bladder (40), wherein direction of a current flowing through the plurality of semiconductors (60) heats or cools designated areas of the wound environment (10); e) a communications module (90), detachable from the communications junction (56), comprising: i) a first face (96) magnetically attracted to the first magnetic field (58); ii) a second face (100) comprising a touchscreen (102); and iii) a computer module (100) comprising one or more of the following components: a microprocessor (150), a memory (152), a graphics unit (154), an audio unit (156) and a transmitter or a transceiver (120); f) a software (180): i) communicating with the group of first sensors (50f); ii) controlling the microprocessor (150), the memory (152), the plurality of semiconductors (60), the graphics unit (154), the audio unit (156), the transmitter or the transceiver (120) and graphics projected by the touchscreen (102), wherein the software (330) controlling the communications module (90) and the computer module (100) is located on the computer module (100), a Cloud server (140), a second computer (130) remote from the computer module (100), or a combination thereof; g) the transmitter or the transceiver (120) adapted for wireless communications, via an available wireless cellular network, an IEEE 802.11 protocol and/or a military protocol, with the Cloud server (140) or the second computer (130) remote from the computer module (100); and h) a circuitry (390) and a power source (360).

Another preferred embodiment of the current invention can be described as a cranial orthotic (20) comprising: a) a bladder (40) positioned proximate to a wound environment (10) and fitted to contact a contour of a portion of an inward side (45) of an exterior shell (22) of the cranial orthotic (20); the bladder (40) comprising: i) a first expansion fluid (36f) mixed with a second expansion fluid (36s) generating a foam volume and a gas supplying a preselected pressure to the wound environment (10); ii) micropores (42) regulating the volume of the gas inside the bladder; and iii) a plurality of polymerized beads (330); b) the exterior shell (22) comprising: i) a removable vent (28) providing ventilation for the wound environment (10) and an access to a communications junction (56); and i) one or more ports (200) extending through the exterior shell (22); the one or more ports (200) adapted to transport the expansion fluids (36f, 36s) to the bladder (40); c) a three dimensional array (48) conforming with the contour of the portion of the inward side (23) of the exterior shell (22) and connected to an inward side (45) of the bladder (40); the three dimensional array comprising: i) a group of first sensors (50f), wherein the group of first sensors (50f) are adapted to sense pressures associated with the wound environment (10); and ii) the communications junction (56) comprising a first magnetic field (58); d) a plurality of semiconductors (60) connected to an inward side (45) of the bladder (40), wherein direction of a current flowing through the plurality of semiconductors (60) heats or cools designated areas of the wound environment (10); e) a communications module (90), detachable from the communications junction (56), comprising: i) a first face (96) magnetically attracted to the first magnetic field (58); ii) a second face (100) comprising a touchscreen (102); and iii) a computer module (100) comprising one or more of the following components: a microprocessor (150), a memory (152), a graphics unit (154), an audio unit (156) and a transmitter or a transceiver (120); f) a software (180): i) communicating with the group of first sensors (50f); ii) controlling the microprocessor (150), the memory (152), the plurality of semiconductors (60), the graphics unit (154), the audio unit (156), the transmitter or the transceiver (120) and graphics projected by the touchscreen (102), wherein the software (330) controlling the communications module (90) and the computer module (100) is located on the computer module (100), a Cloud server (140), a second computer (130) remote from the computer module (100), or a combination thereof; g) the transmitter or the transceiver (120) adapted for wireless communications, via an available wireless cellular network, an IEEE 802.11 protocol and/or a military protocol, with the Cloud server (140) or the second computer (130) remote from the computer module (100); and h) a circuitry (390) and a power source (360).

Still another preferred embodiment of the current invention can be described a cranial orthotic (20) comprising: a) an exterior shell (22) comprising a removable vent (28) providing ventilation for the wound environment (10) and an access to a communications junction (56); b) a three dimensional array (48) conforming with the contour of the portion of the inward side (23) of the exterior shell (22); the three dimensional array comprising: i) a group of first sensors (50f), wherein the group of first sensors (50) are adapted to sense pressures associated with the wound environment (10); and ii) the communications junction (56) comprising a first magnetic field (58); c) a plurality of semiconductors (60) connected to an inward side (23) of exterior shell (22), wherein direction of a current flowing through the plurality of semiconductors (60) heats or cools designated areas of the wound environment (10); d) a communications module (90), detachable from the communications junction (56), comprising: i) a first face (96) magnetically attracted to the first magnetic field (58); ii) a second face (100) comprising a touchscreen (102); and iii) a computer module (100) comprising one or more of the following components: a microprocessor (150), a memory (152), a graphics unit (154), an audio unit (156) and a transmitter or a transceiver (120); e) a software (180): i) communicating with the group of first sensors (50f); ii) controlling the microprocessor (150), the memory (152), the plurality of semiconductors (60), the graphics unit (154), the audio unit (156), the transmitter or the transceiver (120) and graphics projected by the touchscreen (102), wherein the software (330) controlling the communications module (90) and the computer module (100) is located on the computer module (100), a Cloud server (140), a second computer (130) remote from the computer module (100), or a combination thereof; f) the transmitter or the transceiver (120) adapted for wireless communications, via an available wireless cellular network, an IEEE 802.11 protocol and/or a military protocol, with the Cloud server (140) or the second computer (130) remote from the computer module (100); and g) a circuitry (390) and a power source (360).

It is the novel and unique interaction of these simple elements which creates the system within the ambit of the present invention. Pursuant to Title 35 of the United States Code, select preferred embodiments of the current invention follow. However, it is to be understood that the descriptions of the preferred embodiments do not limit the scope of the present invention.

### BRIEF DESCRIPTION OF THE DRAWINGS

FIG. 1 is a perspective of cap or cranial orthotic (20) without bladder (40).

FIG. 2 is a perspective of bladder (40) attachable to cranial orthotic (20).

FIG. 3 portrays a method of creating cured expanded foam (39) for cranial orthotic (20).

FIG. 4 is a perspective of cranial orthotic (20) including bladder (40).

FIG. 4A is a representation of a section of bladder (40) or exterior shell (22w) provided with a plethora of semiconductors (60, 60r, 60f) and the necessary circuitry (390) allowing for operation of the semiconductors. The depiction is applicable to all semiconductors (60, 60r, 60f) associated with cranial orthotic (20).

FIG. 5 is a perspective of three dimensional array (48) of first sensors (50f) and/or second sensors (50s) adapted for fitting into cranial orthotic (20) in view of wound environment (10).

FIG. 6 is a lateral view of an upper portion of cranial orthotic (20) with a portion of cranial orthotic cut away disclosing communications module (90) attached to array (48).

FIG. 7 is perspective of the inside of cap or cranial orthotic (20) including bladder (40) with semiconductors (60) attached thereto.

FIG. 8 is perspective of the inside of cap or cranial orthotic (20) including bladder (40) with semiconductors (60) attached thereto.

FIG. 9 is a perspective of an upper portion of cap or cranial orthotic (20) with communications module (90) attached thereto and array (48) shown in phantom.

FIG. 10 is a top view of bladder (40).

FIGS. 11-18 are view of embodiments of collapsible exterior shell (22) of cranial orthotic (20).

FIG. 18A is a depiction of tie rod (26).

FIG. 18B is a depiction of tie-cord (26) or clamp (26).

FIG. 19 is a depiction of external bag (34) carrying fluids (36f, 38f).

FIG. 19A portrays a top view cross-section of cured foam (39) including tunnels (37).

FIG. 20 portrays planar array (48p).

FIG. 20A portrays array (48, 48p) including circuitry (390) and communications junction (56).

FIG. 20B portrays circuitry (390) and first sensors (50f).

FIG. 21 is a top view of cranial orthotic (20) including exterior shell (22, 22w).

FIG. 22 portrays a rigid semiconductor (60r).

FIG. 23 portrays a flexible semiconductor (60f).

FIG. 24 portrays a substrate (62) provided with carbon nanotubes (66).

FIG. 25 is an exploded view of bottle (34), exterior shell (22) of cranial orthotic (20) and bladder (40) with opening (47) connectable to port (200).

FIG. 26 is a perspective of three dimensional array (48) and semiconductors (60f, 60r) connected to bladder (40).

FIG. 27 is depiction of visual temperature indicator (46) of bladder (40).

FIG. 28 is depiction of second face (100) and touchscreen (102) of communications module (90).

FIG. 28A is an exemplary portrayal of visible touchscreen (102) of communications module (90) that is adapted to show pressures and biometrics associated with wound environment (10) as well as allowing the user to send commands to microprocessor (150) that alter the parameters of wound environment's (10) pressures and biometrics monitored.

FIG. 29 is a perspective of communications module (90).

FIG. 30 is diagrammatic representation of computer module (110).

FIG. 31 is diagrammatic representation of communications networks linking communications module (90), a second computer and a cloud server (140).

### DESCRIPTION OF THE PREFERRED EMBODIMENTS

Although the disclosure hereof is detailed to enable those skilled in the art to practice the invention, the embodiments published herein merely exemplify the present invention.

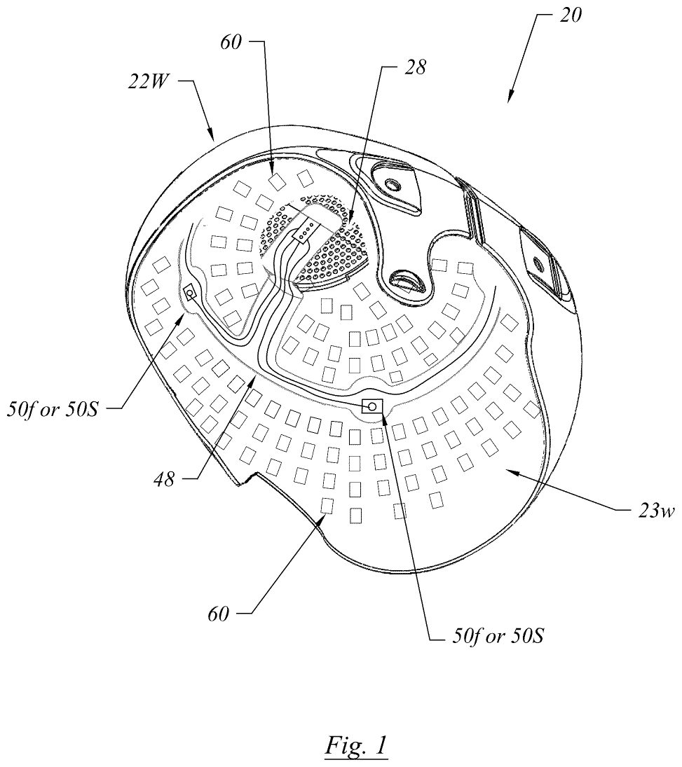

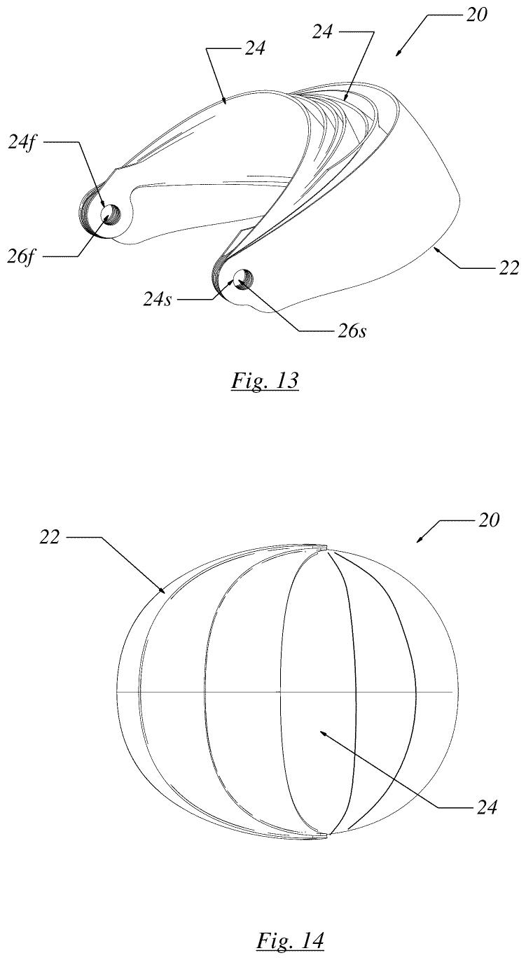

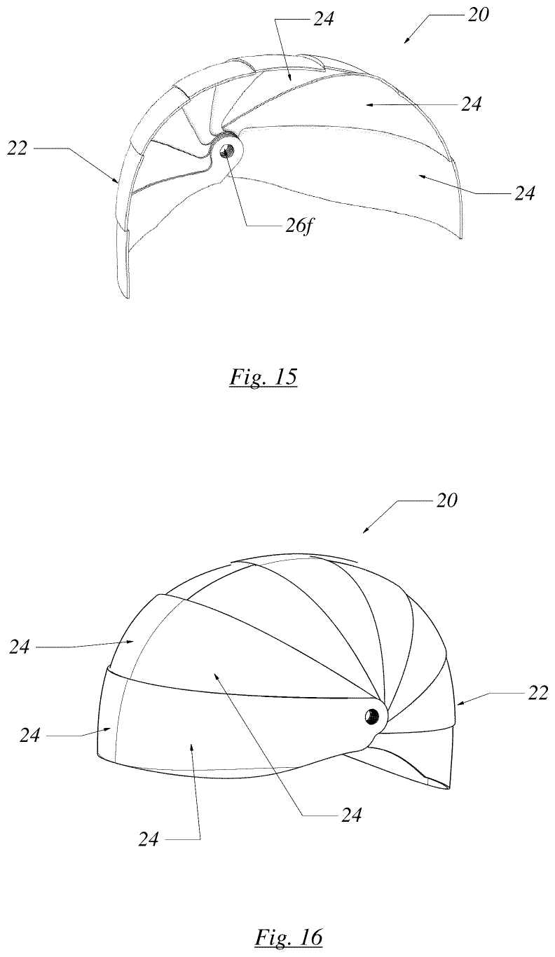

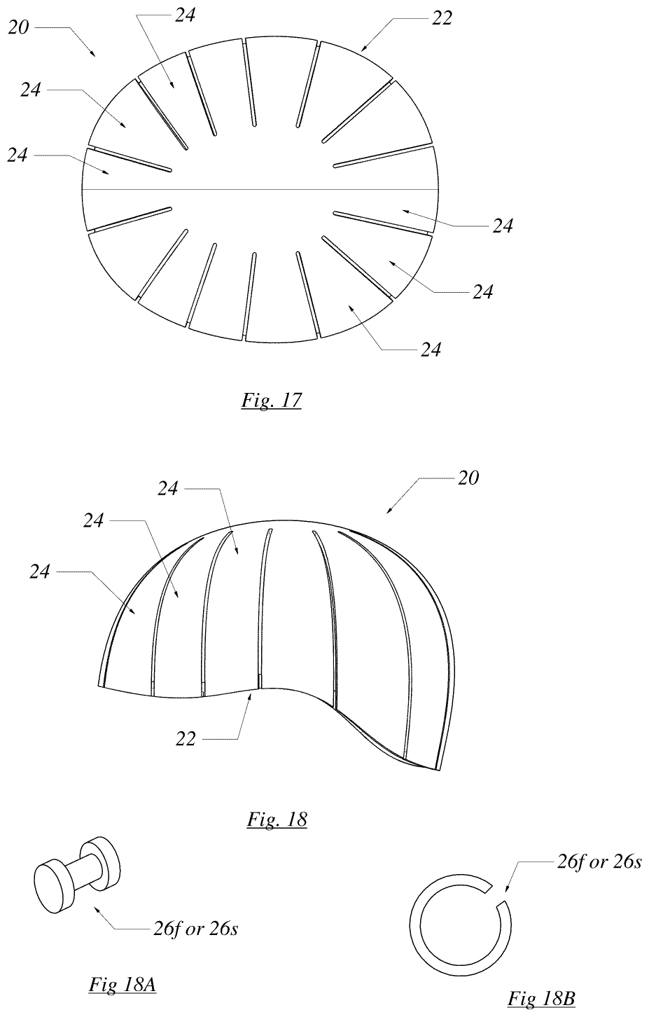

As described above and with reference to FIGS. 1-18, subsequent to a traumatic insult, the present smart custom cranial orthotic (20) can be formed to correspond to a soft tissue or bony defect in the skull or other bone. Preferred embodiments of cranial othotic (20) can include: an uncollapsible exterior shell (22w) without a bladder (40); an uncollapsible exterior shell (22w) with bladder (40) fitted to contact a contour of a portion of an inward side (23w) of the exterior shell (22w); a collapsible exterior shell (22) with bladder (40) fitted to contact a contour of a portion of an inward side (23) of the collapsible exterior shell (22); and a collapsible exterior shell (22) without a bladder (40). Embodiments of cranial orthotic (20) can include helmets, caps and splints.

FIG. 1 is a perspective of cap or helmet (20). Cranial orthotic (20) includes an uncollapsible exterior shell (22w) without a bladder (40). By way of illustration, exterior shell (22s) of cranial orthotic (20) can be constructed of polyurethane, carbon fiber, poly-para-phenylene terephthalamide (Kevlar), polycarbonate, polypropylene, injection molded plastic, high density polyethylene (HDPE), acrylonitrile butadiene styrene (ABS), other polymers and equivalent materials or combinations thereof.

Although not shown in the Drawings, select embodiments of cranial orthotic (20) can be provided with a chin strap. Cranial orthotic (20) includes removable vent (28). Removable vent (28) can improve air movement under helmet (20) to assist with heat control. Removal of vent (28) allows better access to patient's scalp for probes, medication applications, visual observations, and when required, placement of one or more additional sensors without removing helmet (20) from the patient.

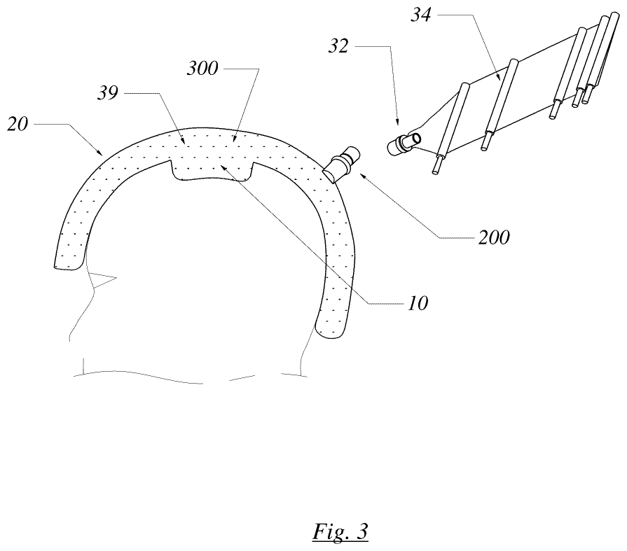

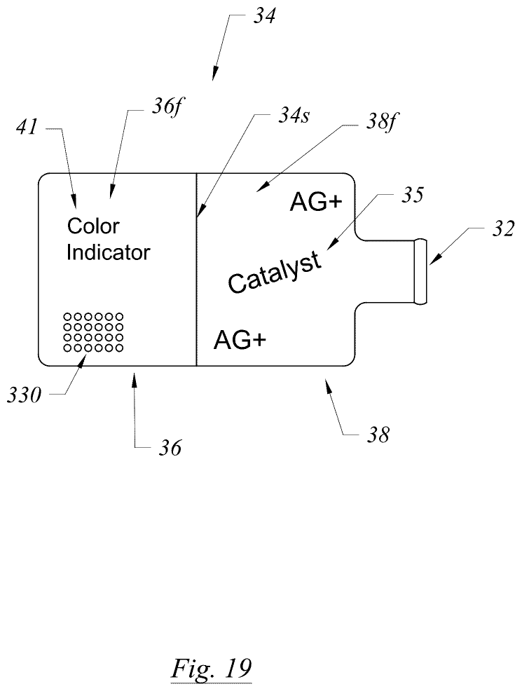

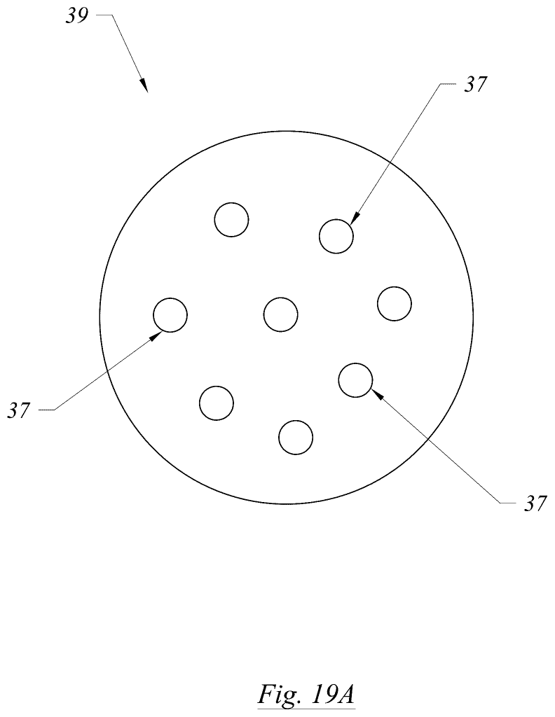

With a view toward FIGS. 3, 19 and 19A, via mouth (32), bag (34) can be connected to port (200) of helmet (20). External packet or bag (34) includes at least two compartmentalized sections (36, 38). Bag (34) is provided with shatterable seal (34s) separating sections (36, 38) and fluids (36f, 38f). When seal (34s) is shattered, fluids (36f, 38f) begin to mix creating a fluid volume that can be injected into the wound environment (10) or bladder (40) via port (200). When the reaction of fluids (36f, 36s) is completed, the fluid volume becomes an expanded or cured foam (39). FIG. 19A portrays a top view cross-section of cured foam (39) including tunnels (37) providing air circulation to wound environment (10). Among other things, extrusion of different volumes of fluids (36f, 36s) from bag (34) allows custom compression on any morphology of the wound environment.

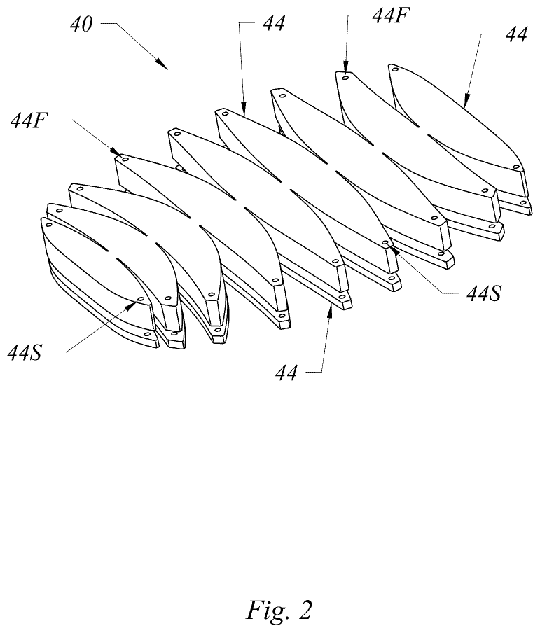

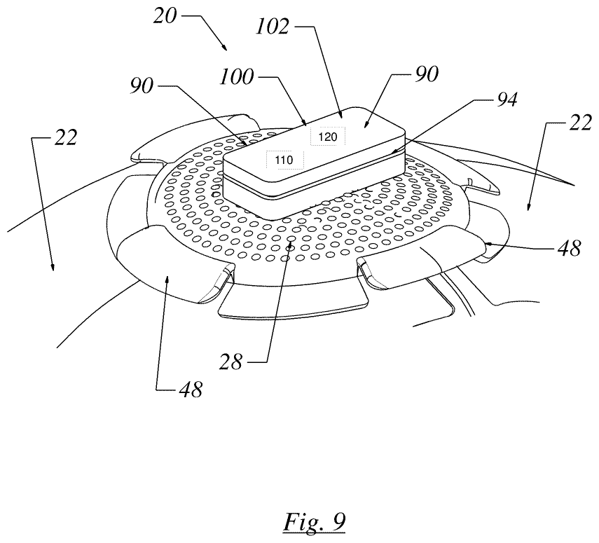

With reference to FIGS. 2 and 4, in use of an embodiment of helmet (20), bladder (40) contacts inward side (23) of exterior shell (22) and can contact the wound environment (10). When helmet (20) is provided with bladder (40), expansion fluids (36f, 38f) can expand about a portion of bladder (40) to create foam (39). Cured foam (39) assists in controlling the pressure applied to the wound environment (10). Bladder (40) can function as an overflow valve for expanded foam (39), that when required can be trimmed and customized subsequent expansion or curing of foam (39). Foaming agents or expansion fluids (36f, 38f) may be composed of silicone, polyurethane, cellulose, bamboo, or other biodegradable agents and will be open cell or an open cell configuration to allow for application of negative pressure or evacuation of fluid or foam (39) when medically required. Foams (39) with open cell configurations allow for therapeutic agents to be applied to the wound environment (10) that can assist with hemostasis and healing. Reactions creating cured foams (39) biocompatible with tissues can generate gas. When medical parameters require, an aerosol spray or mixing-tip gun can be utilized to create cured foams (39) for wound environment (10). It has been discovered that the process creating cured foam (39) and fitting cap or cranial orthotic (20) in situ can be accomplished with a set up time under 10 minutes. Sections (44) of bladder (40) provide for selective zonal adjustment of pressures that can be supplied by adjustments of sections (44) to the wound environment (10). FIG. 10 is a top view of an embodiment of bladder (40).

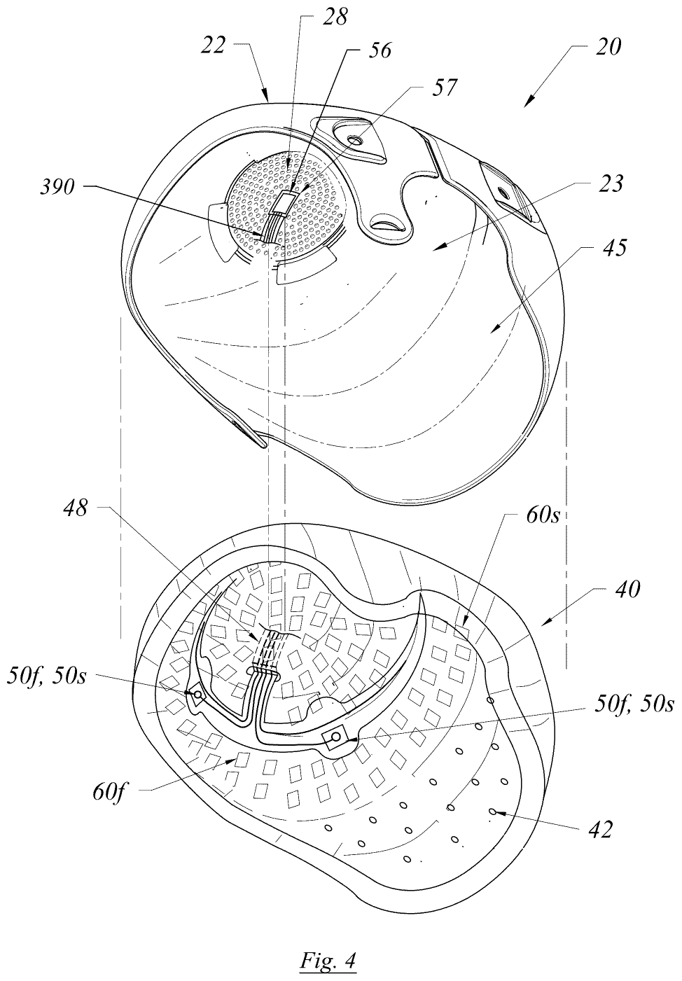

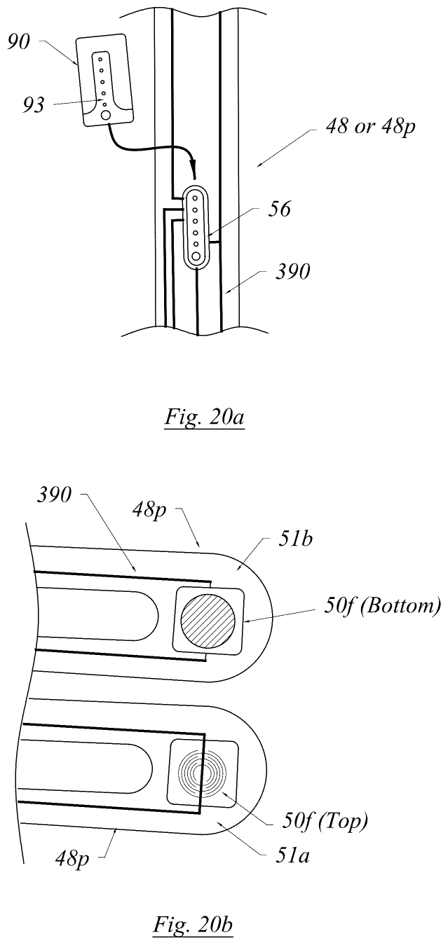

With reference to FIGS. 1, 4, 5 and 20, first sensors (50f) can be positioned on a planar array (48p) or three dimensional array (48). Planar array (48p) is shown in FIG. 20 and a three dimensional array (48) is portrayed in FIG. 5. Regardless of planar array (48p) or three dimensional arrays (48), first sensors (50f) and/or second sensors (50s) function in a similar manner. First sensors (50f) can be dedicated to measuring pressures associated with the wound environment (10). In other embodiments, second sensors (50s), such as piezoresistive, piezoconductive or carbon nanotube sensors, can monitor biometric metabolites and enzymes. Second sensors (50s) can monitor physiological ions such as hydrogen, lactate, potassium, sodium, glucose, galactose, calcium, ketones, chloride, phosphate, bicarbonate, magnesium, etc.

For this Application, with regard to circuitry (390), circuitry (390) includes all circuits, leads, etc. required for the operation of smart cranial orthotic (20).

FIG. 4A is a representation of a section of bladder (40) or exterior shell (22w) provided with a plethora of semiconductors (60, 60r, 60f) and the necessary circuitry (390) allowing for operation of the semiconductors (60, 60r, 60f). The depiction is applicable to all semiconductors (60, 60r, 60f) associated with cranial orthotic (20).

With reference to FIGS. 20, 20A and 20B, planar array (48p) includes communications module (90), first sensors (50f), communications junction (56) and circuitry or leads (390). When medical parameters require, planar array (48p) is adapted for use with cranial orthotic (20). Planar array (48p) can contact or be printed onto inward side (23w) of exterior shell (22w), inward side (23) of exterior shell (22) or bladder (40). Within the ambit of the current invention, communications junction (56) can be provided with a magnetic electroconductive T-pin configuration (93) capable of interconnecting array (48p) with communications module (90). FIG. 20B depicts opposed sides (51a, 51b) of first sensor (50f).

With reference to FIGS. 4, 5, 20A and 20B, cranial orthotic (20) includes array (48) of first sensors (50f) and/or second sensors (50s), communications junction (56) for releasably holding communications module (90) and circuitry or leads (390). Array (48) can contact or be printed onto inward side (23) of exterior shell (22), inward side (23w) of exterior shell (22w) or bladder (40). Circuitry or leads (390) allow intercommunications with first sensors (50f) and/or second sensors (50s) and communications module (90). Within the ambit of the current invention, communications junction (56) can be provided with a magnetic electroconductive T-pin configuration (93) capable of interconnecting array (48) with communications module (90). FIG. 20B depicts oppose sides (51a, 51b) of first sensor (50f).

When medical parameters require, the locations and number of first sensors (50f) positioned on array (48) can be altered and other biometric second sensors (50s) can be incorporated onto/into cranial orthotic (20), bladder (40) or cured fluid (39). Biometric second sensors (50s) can sense biometrics such as pressure, temperature, blood pressure, lactate levels, pH, growth factors, hydrogen levels, sodium levels, potassium level, lactate levels, other electrolytes, cytokines, glucose levels, apoptotic factors, SVO2 and other biometric metric markers.

First sensors (50f) or biometric second sensors (50s) can be piezoresistive, piezoconductive or electroactive inks and printed directly or laminated through materials such as thermoplastic polyurethane (TPU) with an adhesive onto shell (22w) or bladder (40). In another preferred embodiment, array (48) can include carbon nanotubules or multi-channel carbon-nanotubes second sensors (50b) to capture and sense specific ions, including but not limited to, H+, lactate, Na+, K+, glucose, cytokines, growth factors, other electrolytes or biometric markers. One of the fluids (36f, 38f) can include a catalyst (35) and a color indicator (41) to indicate when sufficient foam (39) is created to assist with structural integrity of the wound environment (10). Select embodiments of cranial orthotic (20) will include a visual temperature indicator (46) showing when foam (39) is sufficiently warm to be therapeutic. The temperature indicator (46) can also indicate when semiconductors (60) generate heated or cooled air within a therapeutic or treatable range. Some select embodiments of cranial orthotic (20) will add silver ions to one of the fluids (36f, 38f) to enhance antimicrobial activities.

Cranial orthotic (20) can be provided with bladder (40) positioned proximate to a wound environment (10) and fitted to contact a contour of a portion of an inward side (23) of an unfolded collapsible exterior shell (22) of the cranial orthotic (20). As previously indicated, bag (34) includes at least two compartmentalized sections (36, 38). Once the seal (34s) between the sections (36, 38) is broken, first expansion fluid (36f) is mixed with second expansion fluid (36s) generating a foam volume (39) and a gas that supplies a preselected pressure to the wound environment (10). Micropores (42) assist in regulating the volume of the gas inside bladder (40) and assist in preventing overflow. Select embodiments of first and second fluids (36f, 36s) are provided with polymerized beads (330) that can assist in creating the cured foam (39). It is believed the addition of polymerized beads (330) lessens the weight of cranial orthotic (20) while also assisting in creating airflow channels in the cured foam (39).

With reference to FIGS. 13-16, unfolded collapsible exterior shell (22) is provided with a plurality of interconnected rotatable sections (24). Each rotatable section (24) includes a first aperture (24f) connectable to a first tie (26f) and a second aperture (24s) connectable to the second tie (26f) allowing the plurality of interconnected rotatable sections (24) to collapse about itself when the cranial orthotic (20) is folded. Examples of first and second ties (26f, 26s) include tie-cords, clamps, flexible rods, cerclage or synching ties.

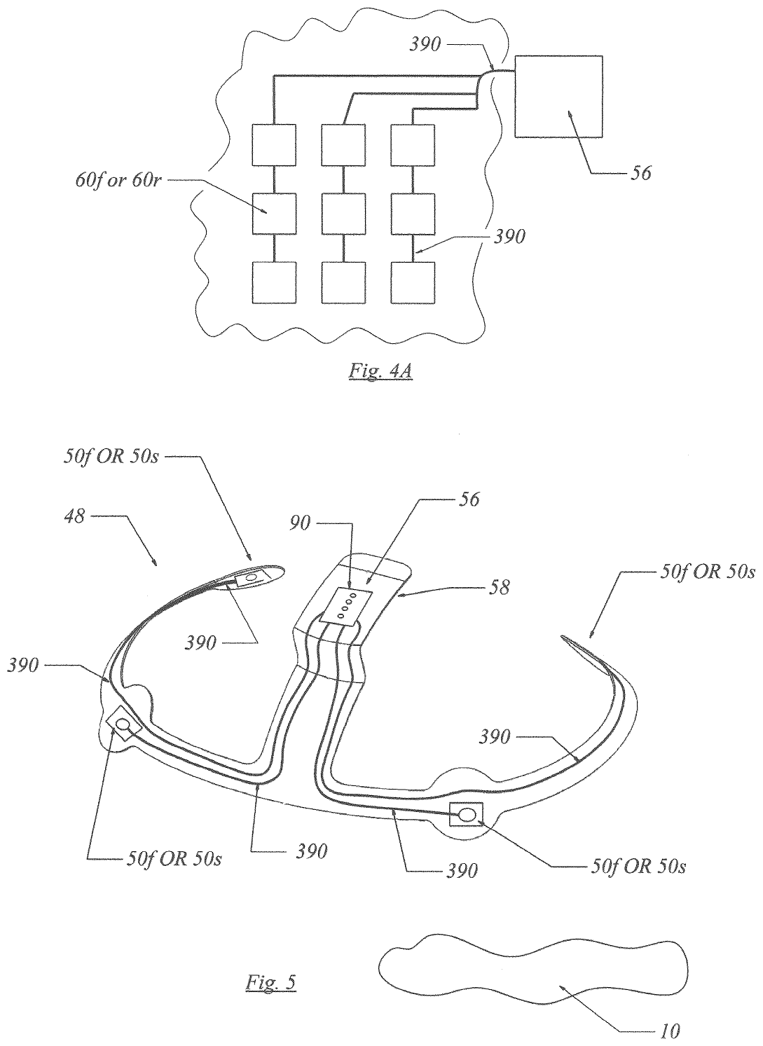

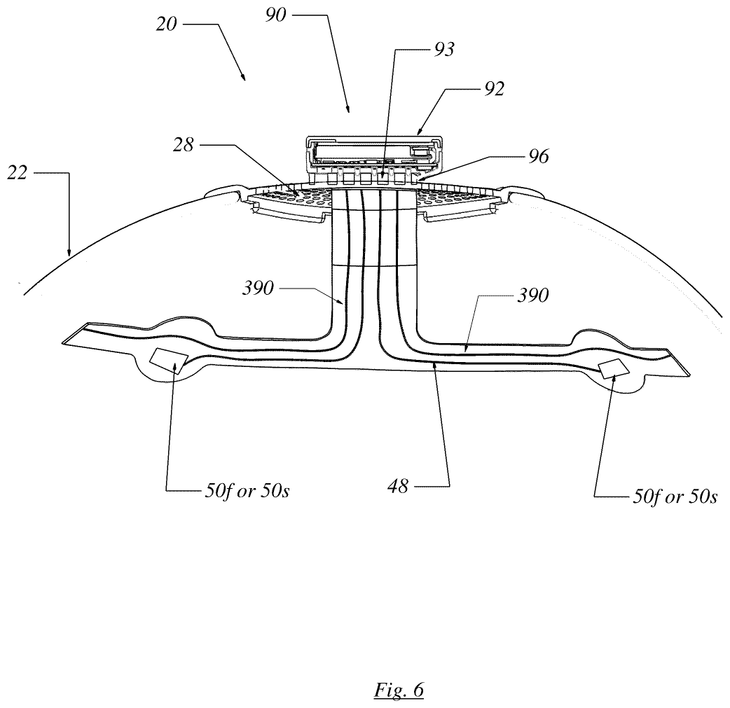

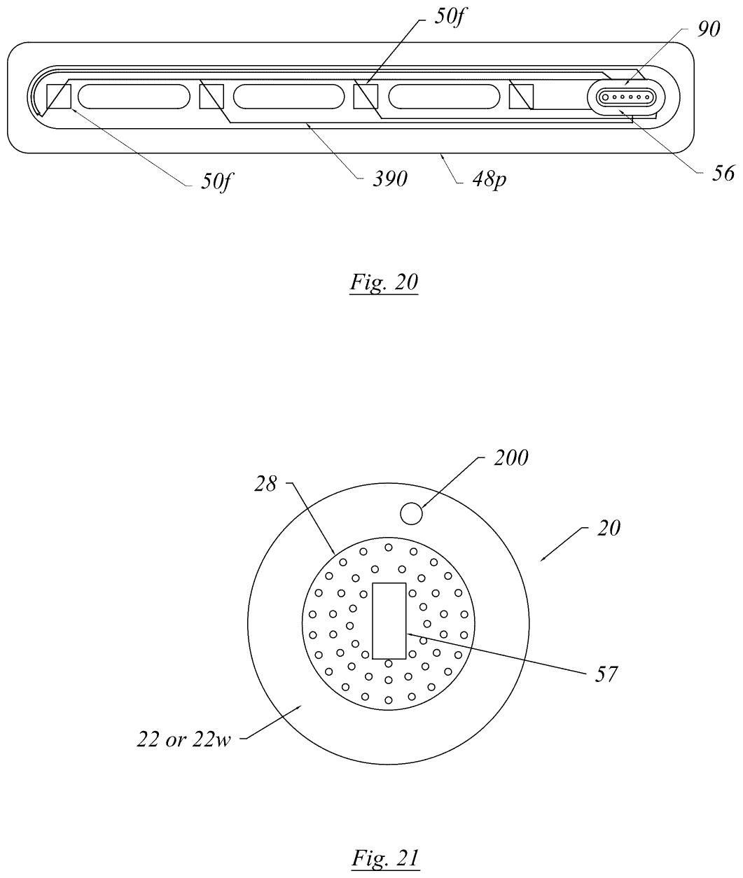

With reference to FIGS. 6 and 21, cranial orthotic (20) includes removable vent (28) providing ventilation for the wound environment (10) and an access to communications junction (56) and one or more ports (200) extending through the exterior shell (22) allowing ports (200) to transport the expansion fluids (36f, 36s) to the bladder (40). Cranial orthotic (20) can also be provided with receptacle (57) connected to communications junction (56) and adapted to receive communications module (90).

With reference to FIGS. 4 and 5, a three dimensional array (48) conforming to the contour of a portion of the inward side (23) of the unfolded collapsible exterior shell (22) is connected to an inward side (23) of bladder (40). Three dimensional array (48) is provided with a group of first sensors (50f) and/or second sensors (50s) and communications junction (56) that includes first magnetic field (58). First sensors (50f) can sense pressures associated with the wound environment (10).

FIG. 21 is a top view of cranial orthotic (20) including exterior shell (22, 22w), receptacle (57 for receiving communications module (90). FIG. 21 also discloses port (200) and removable vent (28).

FIG. 1 portrays a plurality of semiconductors (60) attached to the inward side (23w) of the exterior shell (22w) of cranial orthotic (20). FIG. 4 discloses a plurality of semiconductors (60) connected to an inward side (45) of bladder (40).

Direction of a current flowing through the plurality of semiconductors (60) heats or cools designated areas of the wound environment (10). Depending on the direction of current, the plurality of semiconductors (60) can create up to about a 25 degree Celsius temperature differential between a first temperature and a second temperature of any of the plurality of semiconductors (60). The temperature differential can correspond to each semiconductors' (60) footprints associated with the wound environment (10).

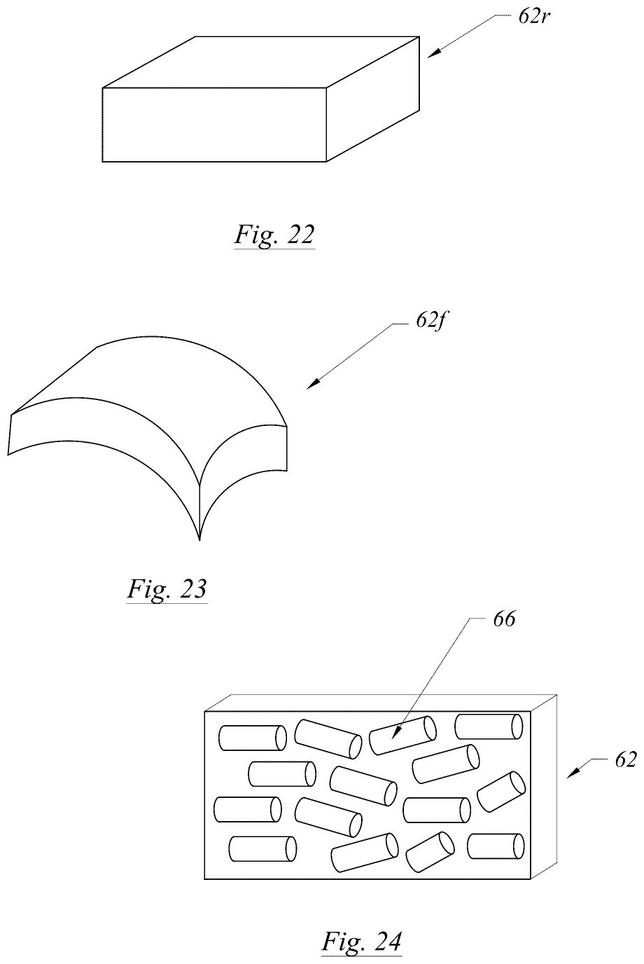

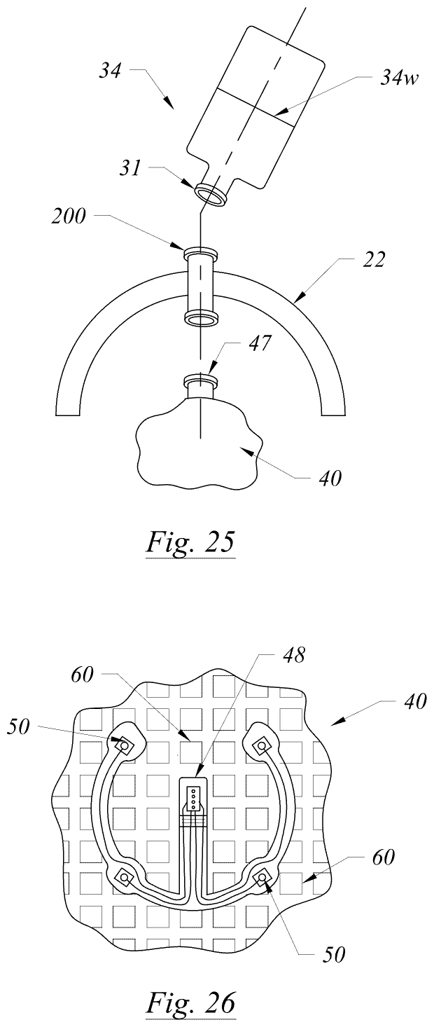

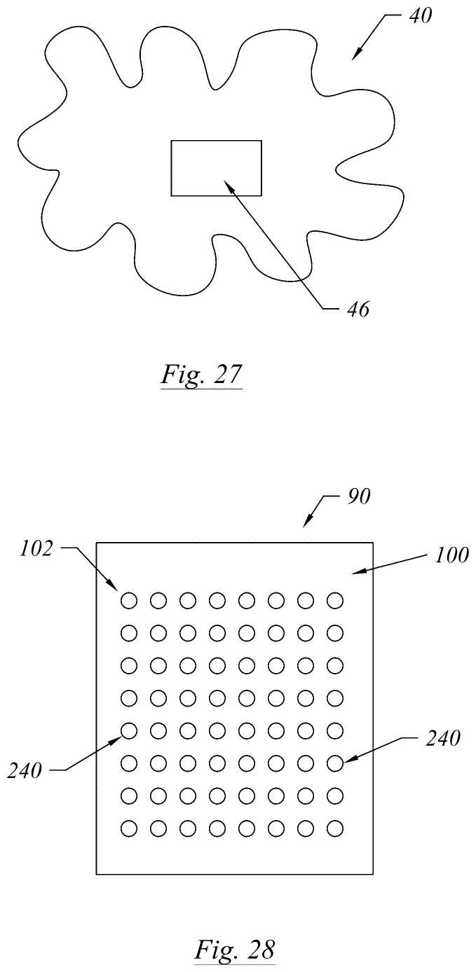

FIGS. 22 and 23 disclose semiconductors or micro-semiconductors that are rigid (60r) or flexible (60f). For example, flexible semiconductors (60f) can be attached to bladder (40). Rigid semiconductors (60r) are manufactured from rigid substrates and flexible semiconductors (60f) are manufactured from flexible substrates such as flexible polymers that can include Alumina Aluminium Nitride or substrates utilized in Miro Peltier modules. When medical conditions require, bladder (40) can include rigid semiconductors (60r).

FIG. 24 portrays a substrate (62) provided with carbon nanotubes (66). Carbon nanotubes (66) can decrease thermal interface resistance. Based on experimentation, it believed that use of carbon nanotubes (66) attached to substrates (62) can improve sensing performance over rigid and flexible micro-semiconductors or semiconductors. Carbon nanotubes (66) can be 3D printed onto rigid or flexible substrates (62r, 62f).

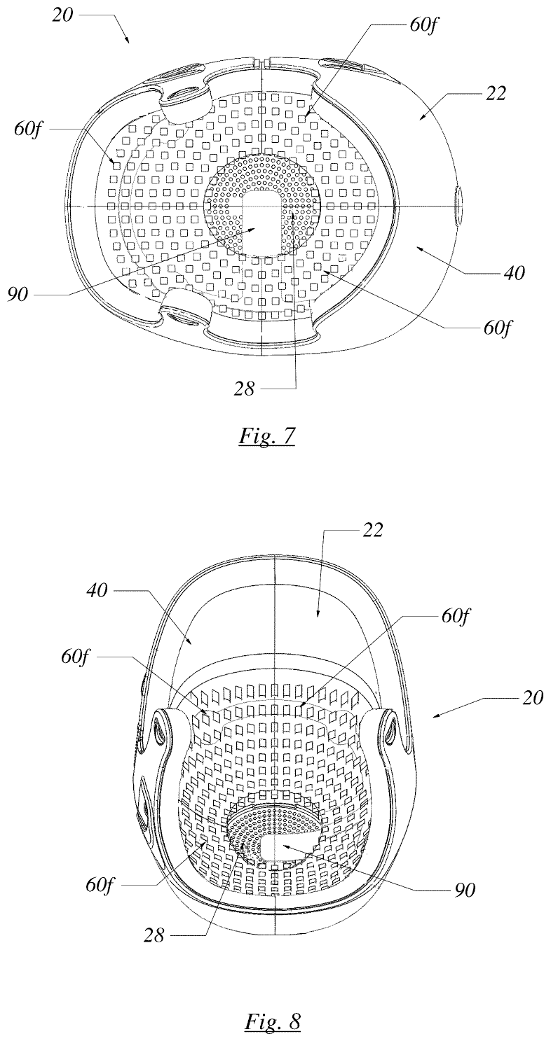

FIGS. 7-8 portray semiconductors (60f, 60r), such as micro-Peltier devices, connected to inner side (23) of exterior shell (22) or bladder (40). Semiconductors (60f, 60r) can have a footprint of less than 2 millimeters², or when medical conditions require, greater than 2 millimeters². The required circuitry (390) or leads interconnects each semiconductor (60f, 60r) to communications module (90) of cranial orthotic (20). In select preferred embodiments, software (180) controls the direction of current flow for each semiconductor that results in semiconductor's (60f, 60r) heating or cooling. In other embodiments, carbon nanotubes (66) can be utilized.

Software (180) can be associated with memory (152) of communications module (90) or at a location remote from cranial orthotic (20). Among other things, software (180) can generate a heat map of temperatures within cranial orthotic (20) to better quantify therapeutic intervention required. Whether in the field or the hospital, it is believed that the selective control of temperatures generated by semiconductors (60f, 60r) inside cranial orthotic (20) can improve medical outcomes for the patient.

Communications module (90) can be provided with computer module (110). Computer module (110) can be provided with one or more of the following components: a microprocessor (150), a memory (152), a graphics unit (154), an audio unit (156) and a transmitter or a transceiver (120).

Communications module (90) is detachable from the communications junction (56). Communications module (90) includes first face (96) magnetically attracted to the first magnetic field (58) and second face (100) including a touchscreen (102). Via leads or circuitry (390), computer module (110) is connected to communications junction (56), first and second sensors (50f, 50s) and semiconductors (60r, 60f). In select preferred embodiments, computer module (110) can be connected to T-pins (93).

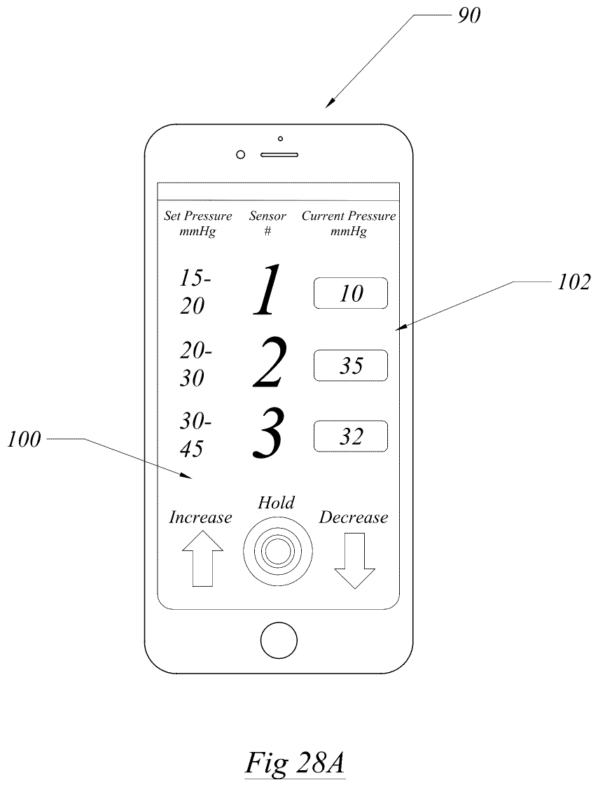

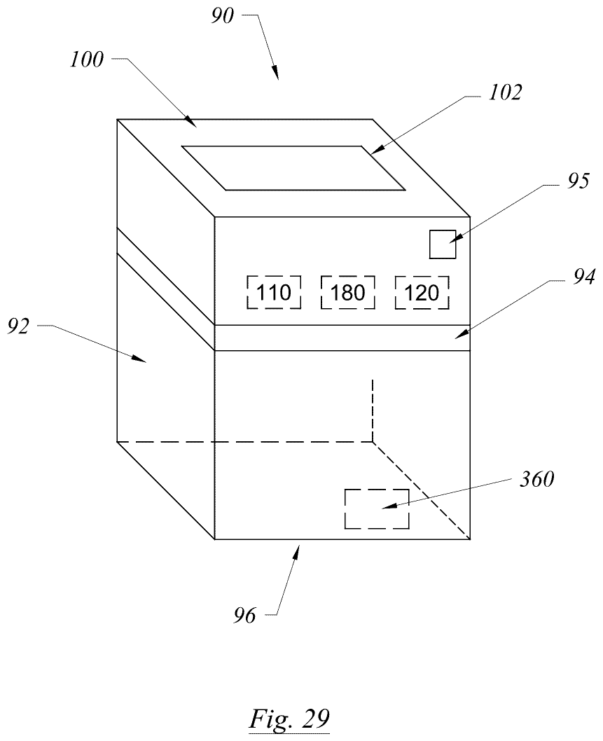

With reference to FIG. 28, software (180) and computer module (110) can cause LEDs (240) beneath touchscreen (102) to show different colors symbolizing the condition of the wound environment (10). For example, green is normal, blue is too low and red it too high. Software (180), computer module (110) and LEDs (240) can convert pressures and other biometrics associated with first sensors (50f), second sensors (50s) and semiconductors (60f, 60r) into numerical or color graphics visible on touchscreen (102). Graphics display measurable medical parameters alerting the user of cranial orthotic (20) of steady state, normal or abnormal conditions associated with the wound environment (10).

FIG. 28A is an exemplary portrayal of visible touchscreen (102) of communications module (90) that is adapted to show pressures and biometrics associated with wound environment (10). Additionally, touchscreen (102) allows the user to send commands to microprocessor (150) that alter the parameters of wound environment's (10) pressures and biometrics supplied/monitored by cranial orthotic (20).

Communications module (90) includes housing (92). Alarms can be audible or visual. Audible alert is generated by speaker (95) and visual alert can be created by LED band (94) positioned on outward side of housing (92) or LEDs (240) associated with touchscreen (102). The alarms are activated when a sensed pressure or biometric for the patient is outside of a predetermined range. The LED band (94) changes colors when the sensed pressure or biometric is outside the predetermined range. By way of illustration, LED (94) can turn red when the pressures in cranial orthotic (20) are calculated to be excessive or blue to designate cranial orthotic (20) is incorrectly fitted, positioned or at risk for movement.

Housing (92) is provided with first face (96) adapted to be received by junction (56) including first magnetic source (58) and receptacle (57). In select preferred embodiments, a magnetic attraction between first face (96) and junction (56) assist in securing electrical connections between first face (96) and junction (56). As previously indicated, junction (56) can include a magnetic electroconductive T-pins (93) connected to first face (96). Housing (92) can be provided with a tongue, rail or other device to assist in attaching housing (92) to shell (22) of cranial orthotic (20).

Housing (92) can be provided with second or outward or second face (100) visible by the user of communications module (90). An OLED or AMOLED touch screen (102) can be incorporated into outward face (100) to provide user access to communications module's (90) computer module (110) can be provided with one or more of the following components: microprocessor (150), memory (152), visual graphics unit (154), audio unit (156), transmitter or transceiver (120), circuitry interconnecting the components and software (180) for controlling the components and cranial orthotic (20). Among other things, computer module (110) can calculate pressures and biometrics sensed by sensors (50f, 50s), control current flow to semiconductors (60) and generate displays of data on touchscreen (102). Within the scope of the current invention, touchscreen (102) can display data correlated and sensed by sensors (50f, 50s) and semiconductors (60). The visualized, calculated and correlated data can be supplied to detachable communications module (90), cloud server (140) or second computer (130) remote from communications module (90) or a combination thereof allowing the user of touchscreen (102) to control application of pressure to wound environment (10). Pressures supplied to wound environment (10) can also be controlled by cloud server (140) or second computer remote (130) remote from communications module (90). Touchscreen (102) can also display correlated data from biometric sensors (50) sensing one or more of pressure, temperature, blood pressure, lactate levels, pH, growth factors, hydrogen levels, sodium levels, potassium level, lactate levels, other electrolytes, cytokines, glucose levels, apoptotic factors or SVO2.

Software (180) communicates with the group of first sensors (50f), second sensors (50s) and semiconductors (60) and controls microprocessor (150), memory (152), graphics unit (154), audio unit (156), transmitter or transceiver (120) and graphics projected by touchscreen (102). Within the scope of the current cranial orthotic (20), the software (180) controlling the communications module (90) and the computer module (110) can be located on the computer module (110), a Cloud server (140), a second computer (130) remote from the computer module (110), or a combination thereof.

Transmitter or transceiver (120) is adapted for wireless communications, via an available wireless cellular network, an IEEE 802.11 protocol and/or military protocols, with Cloud server (140) or second computer (130) remote from the computer module (110).

Cranial orthotic (20) is adapted for wireless communications via any wireless network such as available cellular networks, IEEE 802.11 protocol at a frequency of 2.4 GHz (Wi-Fi or Bluetooth) and/or military protocols. Transmitter or transceiver (120) can communicate with a Cloud server (140) or second computer (130) such as a mobile computing device (smart phone, tablet, etc.) or a personal computer or any type of computing device remote from orthotic (20). Among other things, devices remote from cranial orthotic (20) can be utilized to store data, calculate biometrics and/or control pressures applied by cranial orthotic (20) to the wound environment (10). For select preferred embodiments of sensors (50f, 50s), one or more sensors (50f, 50s) can be equipped with wireless capabilities to communicate wirelessly with communications module (90), cloud server or second computer (130).

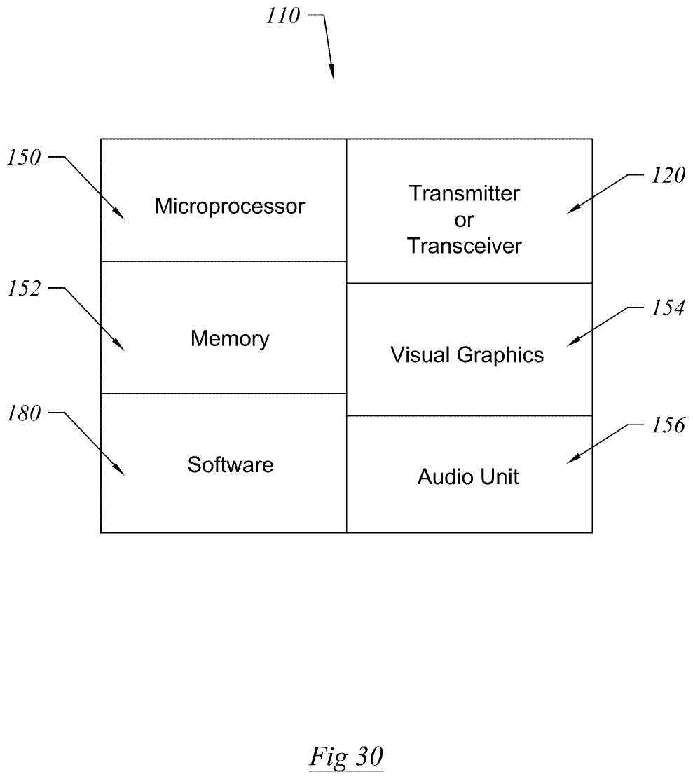

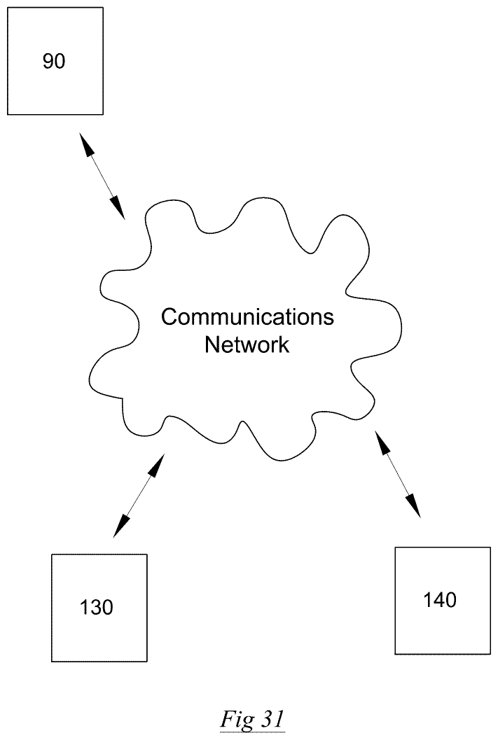

As shown in FIG. 30, computer module (110), microprocessor (150), memory (152), visual graphics unit (154), audio unit (156), transmitter or transceiver (120) and software (180) adapted for cranial orthotic (20) are represented diagrammatically. Those skilled in the art understand cranial orthotic (20) includes the necessary circuitry or leads (390) and interconnections for its functionality.

Power sources (360) for communications module (90) include but are not limited to rechargeable sources such as lithium ion, lithium iron phosphate, solid state, tab-less batteries or other usable power sources. Within the scope of the invention, power source (360) can be attached to housing (92). Rechargeable power sources (360) can be detachable from housing (92) to engage the recharging energy supply.

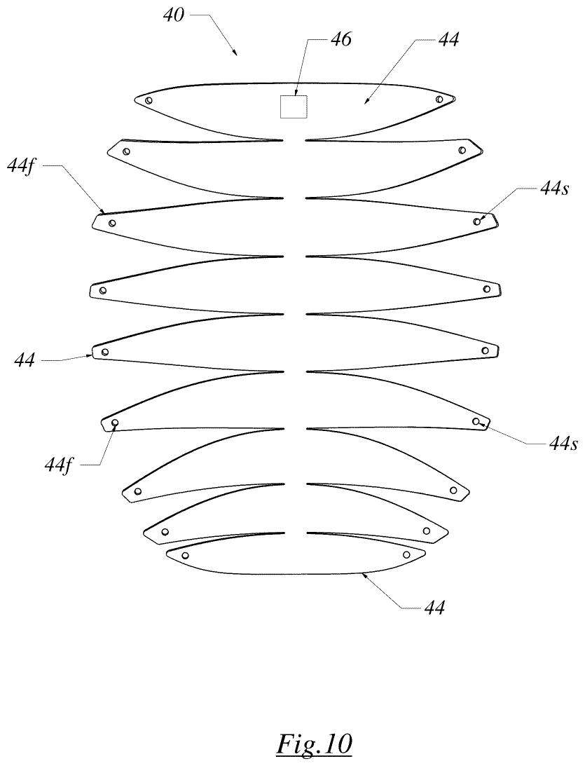

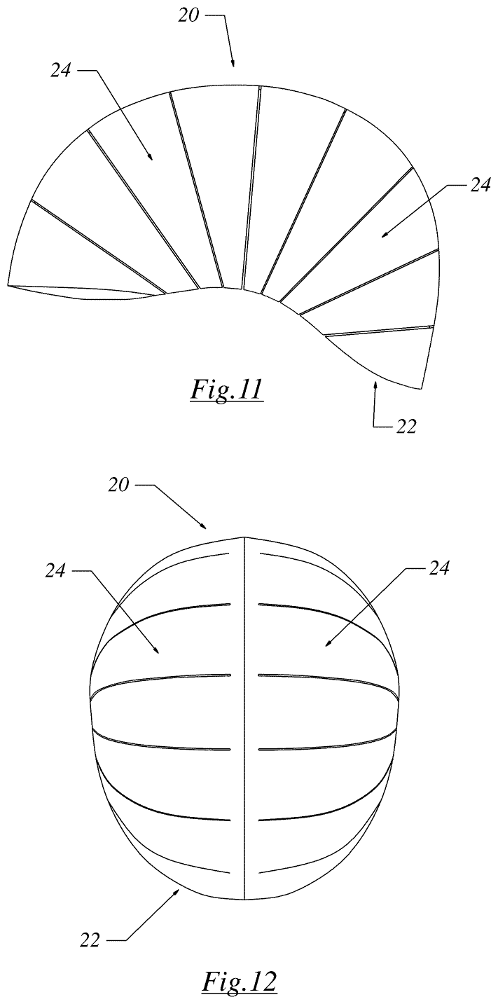

FIGS. 11-18 disclose different shells (22) of the current cranial orthotic (20). Sections (24) are collapsible and can be flattened for more efficient storage. Among the many potential configurations for shells (22), gore map design, armadillo or nautilus telescoping segments and tulip flanges are portrayed. Although not shown, draw strings, zip ties, laces, hook and loop fasteners and Boa-type clip technology can assist with rapid formation of the shape of the cranial orthotic (20).

Select preferred embodiments of the current invention have been disclosed and enabled as required by Title 35 of the United States Code.

What is claimed is:

1. A cranial orthotic comprising:
a) a bladder positioned proximate to a wound environment and fitted to contact a contour of a portion of an inward side of an unfolded collapsible exterior shell of the cranial orthotic; the bladder comprising:
i) a first expansion fluid mixed with a second expansion fluid generating a foam volume and a gas supplying a preselected pressure to the wound environment;
ii) micropores regulating the volume of the gas inside the bladder; and
iii) a plurality of polymerized beads;
b) the unfolded collapsible exterior shell comprising:
i) a plurality of interconnected rotatable sections; each rotatable section comprising a first aperture connected to a first tie and a second aperture connected to the second tie such that the plurality of interconnected rotatable sections is collapsible about itself when the cranial orthotic is folded;
ii) a removable vent providing ventilation for the wound environment and an access to a communications junction; and
iii) one or more ports extending through the exterior shell; the one or more ports adapted to transport the expansion fluids to the bladder;
c) a three dimensional array conforming with the contour of the portion of the inward side of the unfolded collapsible exterior shell and connected to an inward side of the bladder; the three dimensional array comprising:
i) a group of first sensors, wherein the group of first sensors (50) are adapted to sense pressures associated with the wound environment; and
ii) the communications junction comprising a first magnetic field;
d) a plurality of semiconductors connected to an inward side of the bladder, wherein direction of a current flowing through the plurality of semiconductors heats or cools designated areas of the wound environment;
e) a communications module, detachable from the communications junction, comprising:
i) a first face magnetically attracted to the first magnetic field;
ii) a second face comprising a touchscreen; and
iii) a computer module comprising one or more of the following components: a microprocessor, a memory, a graphics unit, an audio unit and a transmitter or a transceiver;
f) a software:
i) communicating with the group of first sensors;
ii) controlling the microprocessor, the memory, the plurality of semiconductors, the graphics unit, the audio unit, the transmitter or the transceiver and graphics projected by the touchscreen, wherein the software controlling the communications module and the computer module is located on the computer module, a Cloud server, a second computer remote from the computer module, or a combination thereof;
g) the transmitter or the transceiver adapted for wireless communications, via an available wireless cellular network, an IEEE 802.11 protocol and/or a military protocol, with the Cloud server or the second computer remote from the computer module; and
h) a circuitry and a power source.

2. The cranial orthotic of claim 1, wherein up to about a 25 degree Celsius temperature differential is generated between a first temperature and a second temperature of any of the plurality of semiconductors receiving a current.

3. The cranial orthotic of claim 2 comprising a second group of sensors sensing one or more second biometrics of temperature, blood pressure, lactate levels, pH, growth factors, hydrogen levels, sodium levels, potassium level, lactate levels, other electrolytes, cytokines, glucose levels, apoptotic factors, nitric oxide levels or SVO2.

4. The cranial orthotic of claim 3 further comprising:
a) a visual or audible alarm or a combination thereof activated when the sensed pressure or second biometric for a patient is outside of a predetermined range;
b) a first visual indicator indicating a catalytic reaction is occurring with the first expansion fluid and the second expansion fluid; and
c) a second visual indicator indicating the temperature of the cured foam has increased.

5. The cranial orthotic of claim 3, wherein a substrate comprises carbon nanotubes.

6. The cranial orthotic of claim 5 further comprising tunnels.

7. The cranial orthotic of claim 6, wherein the graphics displays a pressure or a temperature or both associated with the wound environment.

8. The cranial orthotic of claim 5, wherein the semiconductors are rigid or flexible or a combination thereof.

9. A cranial orthotic comprising:
a) a bladder positioned proximate to a wound environment and fitted to contact a contour of a portion of an inward side of an exterior shell of the cranial orthotic; the bladder comprising:
i) a first expansion fluid mixed with a second expansion fluid generating a foam volume and a gas supplying a preselected pressure to the wound environment;
ii) micropores regulating the volume of the gas inside the bladder; and
iii) a plurality of polymerized beads;
b) the exterior shell comprising:
i) a removable vent providing ventilation for the wound environment (10) and an access to a communications junction; and
i) one or more ports extending through the exterior shell; the one or more ports adapted to transport the expansion fluids to the bladder;
c) a three dimensional array conforming with the contour of the portion of the inward side of the exterior shell and connected to an inward side of the bladder; the three dimensional array comprising:
i) a group of first sensors, wherein the group of first sensors are adapted to sense pressures associated with the wound environment; and
ii) the communications junction comprising a first magnetic field;
d) a plurality of semiconductors connected to an inward side of the bladder, wherein direction of a current flowing through the plurality of semiconductors heats or cools designated areas of the wound environment;
e) a communications module, detachable from the communications junction, comprising:
i) a first face magnetically attracted to the first magnetic field;
ii) a second face comprising a touchscreen; and
iii) a computer module comprising one or more of the following components: a microprocessor, a memory, a graphics unit, an audio unit and a transmitter or a transceiver;
f) a software:
i) communicating with the group of first sensors;
ii) controlling the microprocessor, the memory, the plurality of semiconductors, the graphics unit, the audio unit, the transmitter or the transceiver and graphics projected by the touchscreen, wherein the software controlling the communications module and the computer module is located on the computer module, a Cloud server, a second computer remote from the computer module, or a combination thereof;
g) the transmitter or the transceiver adapted for wireless communications, via an available wireless cellular network, an IEEE 802.11 protocol and/or a military protocol, with the Cloud server or the second computer remote from the computer module; and
h) a circuitry and a power source.

10. The cranial orthotic of claim 9, wherein up to about a 25 degree Celsius temperature differential is generated between a first temperature and a second temperature of any of the plurality of semiconductors receiving a current.

11. The cranial orthotic of claim 10 comprising a second group of sensors sensing one or more second biometrics of pressure, temperature, blood pressure, lactate levels, pH, growth factors, hydrogen levels, sodium levels, potassium level, lactate levels, other electrolytes, cytokines, glucose levels, apoptotic factors, nitric oxide levels or SVO2.

12. The cranial orthotic of claim 11 further comprising:
a) a visual or audible an alarm or a combination thereof when the sensed pressure or other second biometric for a patient is outside of a predetermined range;
b) a first visual indicator indicating a catalytic reaction is occurring with the first expansion fluid and the second expansion fluid; and
c) a second visual indicator indicating the temperature of the cured foam has increased.

13. The cranial orthotic of claim 12, wherein a substrate comprises carbon nanotubes.

14. The cranial orthotic of claim 13 further comprising tunnels.

15. The cranial orthotic of claim 14, wherein the semiconductors are rigid or flexible or a combination thereof.

16. A cranial orthotic comprising:
a) an exterior shell comprising a removable vent providing ventilation for the wound environment and an access to a communications junction;
b) a three dimensional array conforming with the contour of the portion of the inward side of the exterior shell; the three dimensional array comprising:
i) a group of first sensors, wherein the group of first sensors are adapted to sense pressures associated with the wound environment; and
ii) the communications junction comprising a first magnetic field;
c) a plurality of semiconductors connected to an inward side of exterior shell, wherein direction of a current flowing through the plurality of semiconductors heats or cools designated areas of the wound environment;
d) a communications module, detachable from the communications junction, comprising:
i) a first face magnetically attracted to the first magnetic field;
ii) a second face comprising a touchscreen; and
iii) a computer module comprising one or more of the following components: a microprocessor, a memory, a graphics unit, an audio unit and a transmitter or a transceiver;
e) a software:
i) communicating with the group of first sensors;
ii) controlling the microprocessor, the memory, the plurality of semiconductors, the graphics unit, the audio unit, the transmitter or the transceiver and graphics projected by the touchscreen, wherein the software controlling the communications module and the computer module is located on the computer module, a Cloud server, a second computer remote from the computer module, or a combination thereof;
f) the transmitter or the transceiver adapted for wireless communications, via an available wireless cellular network, an IEEE 802.11 protocol and/or a military protocol, with the Cloud server or the second computer remote from the computer module; and
g) a circuitry and a power source.

17. The cranial orthotic of claim 16, wherein up to about a 25 degree Celsius temperature differential is generated between a first temperature and a second temperature of any of the plurality of semiconductors receiving a current.

18. The cranial orthotic of claim 17 comprising a second group of sensors (50s) sensing one or more second biometrics of, temperature, blood pressure, lactate levels, pH, growth factors, hydrogen levels, sodium levels, potassium level, lactate levels, other electrolytes, cytokines, glucose levels, apoptotic factors, nitric oxide levels or SVO2.

19. The cranial orthotic of claim 18 further comprising:
a) a visual or audible alarm or a combination thereof activated when the sensed pressure or second biometric for a patient is outside of a predetermined range;
b) a first visual indicator indicating a catalytic reaction is occurring with the first expansion fluid and the second expansion fluid; and
c) a second visual indicator indicating the temperature of the cured foam has increased.

20. The cranial orthotic of claim 19, wherein a substrate comprises carbon nanotubes.

21. The cranial orthotic of claim 20, wherein the semiconductors are rigid or flexible or a combination thereof.
